(19) United States
(12) Patent Application Publication
(10) Pub. No.: US 2024/0165298 A1
(43) Pub. Date: May 23, 2024

(54) 3D PRINTING BONE-REGENERATION SCAFFOLDS COMPOSED OF BIOLOGICALLY-DERIVED BONE POWDER

(71) Applicant: COLORADO STATE UNIVERSITY RESEARCH FOUNDATION, Fort Collins, CO (US)

(72) Inventors: David PRAWEL, Fort Collins, CO (US); Claire BAILEY, Colorado Springs, CO (US); Genesis MARRERO, Fort Collins, CO (US)

(21) Appl. No.: 18/283,587

(22) PCT Filed: Mar. 23, 2022
(86) PCT No.: PCT/US2022/021536

§ 371 Date: Sep. 22, 2023

Related U.S. Application Data
(60) Provisional application No. 63/164,722, filed on Mar. 23, 2021.

(51) Int. Cl. A61L 27/22; A61L 27/36

(52) U.S. Cl. CPC A61L 27/22; A61L 27/3608; A61L 27/3616; A61L 2300/414; A61L 2430/02

(57) ABSTRACT

A method for fabricating a bone-regeneration scaffold may include providing a printing material including a biologically-derived bone powder, and fabricating, via a 3D printer, the bone-regeneration scaffold using the printing material.

### CROSS-REFERENCE TO RELATED APPLICATIONS

This application claims the benefit of, and priority to, U.S. Provisional Patent Application No. 63/164,722, filed on Mar. 23, 2021, and titled “3D Printing Bone-Regeneration Scaffolds Composed of Bone Powder,” the disclosure of which is expressly incorporated herein by reference in its entirety.

### FIELD OF THE DISCLOSURE

The present disclosure relates generally to bone-regeneration scaffolds for treating bone defects of human or veterinary patients and more particularly to bone-regeneration scaffolds or other devices composed of biologically-derived bone powder and methods for fabricating such bone-regeneration scaffolds or devices by 3D printing.

### BACKGROUND OF THE DISCLOSURE

Tissue engineered solutions have emerged that deploy biodegradable, osteoconductive scaffolds, and bioactive agents to provide osteoinductive stimulus to support osseointegration, with suitable porosity to enable nutrient and waste exchange and angiogenesis. See Viateau, V. et al. Long-bone critical-size defects treated with tissue-engineered grafts: A study on sheep. J Orthop Res 2007; 25:741-9. https://doi.org/10.1002/jor.20352; Vidal, L. et al. Reconstruction of Large Skeletal Defects: Current Clinical Therapeutic Strategies and Future Directions Using 3D Printing. Front Bioeng Biotechnol 2020; 8:61. https://doi.org/10.3389/fbioe.2020.00061; Bhumiratana, S. et al. Tissue-engineered autologous grafts for facial bone reconstruction. Sci Transl Med 2016; 8:343ra83-343ra83. https://doi.org/10.1126/scitranslmed.aad5904. Ceramic materials, which may be based on calcium phosphate such has hydroxyapatite (HAp) and beta-tri-calcium phosphate ((3-TCP), are widely used for bone regeneration scaffolds (see Bose, S. et al. Additive manufacturing of biomaterials. Progress in Materials Science 2018; 93:45-111. https://doi.org/10.1016/j.pmatsci.2017.08.003; Vorndran et al. 3D printing of ceramic implants. MRS Bulletin 2016; 41:71. https://doi.org/10.1557/mrs.2015.326) and are approved by the FDA for use in many applications. These materials demonstrate high levels of bioactivity (osteoinduction, osteoconduction and osteointegration), making them well suited for bone regeneration scaffolds. β-TCP is an excellent and widely used biomaterial for bone regeneration but provides insufficient mechanical support for adequate load bearing. β-TCP is completely bioreplaceable by new, native bone. It releases calcium during degradation, which supports bone formation, resulting in excellent osteoconductivity. See Vorndran et al. However, despite excellent bone regeneration properties, success of these scaffolds is hampered by inadequate structural properties required for human-scale load-bearing. See Bose, S. et al. They are simply too brittle to serve well as scaffolds.

Polycaprolactone (PCL) is a widely used polymeric biomaterial (see Lu, L. et al. Biocompatibility and biodegradation studies of PCL/β-TCP bone tissue scaffold fabricated by structural porogen method. J Mater Sci: Mater Med 2012; 23:2217-26. https://doi.org/10.1007/s10856-012-4695-2; Huang, B. et al. Polymer-Ceramic Composite Scaffolds—The Effect of Hydroxyapatite and β-tri-Calcium Phosphate. Materials 2018; 11:129. https://doi.org/10.3390/ma11010129; Woodruff, M A. et al. The return of a forgotten polymer—Polycaprolactone in the 21st century. Progress in Polymer Science 2010; 35:1217-56. https://doi.org/10.1016/j.progpolymsci.2010.04.002.) due to its excellent biocompatibility, and is FDA approved for medical use. PCL is popular in bone tissue engineering due to its relatively long, controllable (see Woodruff, M A. et al.) degradation rate in vivo, mechanical strength and elasticity (see Woodruff, M A. et al.; Eshraghi S. et al. Mechanical and microstructural properties of polycaprolactone scaffolds with one-dimensional, two-dimensional, and three-dimensional orthogonally oriented porous architectures produced by selective laser sintering. Acta Biomaterialia 2010; 6:2467-76. https://doi.org/10.1016/j.actbio.2010.02.002; Brunello G. et al. Powder-based 3D printing for bone tissue engineering. Biotechnology Advances 2016; 34:740-53. https://doi.org/10.1016/j.biotechadv.2016.03.009; Jiang L. et al. Biodegradable Polymers and Polymer Blends. Handbook of Biopolymers and Biodegradable Plastics. Elsevier; 2013. pp. 109-28). PCL is also radiolucent, enabling real-time radiographic assessment. See Choi S. et al. New clinical application of three dimensional-printed polycaprolactone/β-tricalcium phosphate scaffold as an alternative to allograft bone for limb-sparing surgery in a dog with distal radial osteosarcoma. The Journal of Veterinary Medical Science 2019; 81:434-9. https://doi.org/10.1292/jvms.18-0158. However, PCL is hydrophobic and demonstrates poor osteoconduction and osteoinduction. See Huang, B. et al.; Woodruff, M A. et al. Attempts to improve bioactivity of PCL by blending with β-TCP have some success (see Bose, S. et al.; Woodruff, M A. et al.), but the resulting scaffolds remain too weak to support human-scale loads, with lower bioactivity than β-TCP alone.

Many researchers attempt to make calcium phosphate-based scaffolds stronger by adding small amounts of inorganic materials like silica and M and Sn. These additional materials seem to help improve the toughness and ductility, apparently by acting as crack stoppers in the very brittle materials. It is hypothesized that these additives work because they have similarities with minerals naturally found in human bone.

There remains a need for improved bone-regeneration scaffolds and methods of forming bone-regeneration scaffolds for human or veterinary patients, which may overcome one or more of the drawbacks associated with existing technology.

### SUMMARY OF THE DISCLOSURE

The present disclosure provides bone-regeneration scaffolds or other devices composed of biologically-derived bone powder and methods for fabricating such bone-regeneration scaffolds by 3D printing. In one aspect, a method for fabricating a bone-regeneration scaffold is provided. In one embodiment, the method may include providing a printing material including a biologically-derived bone powder, and fabricating, via a 3D printer, the bone-regeneration scaffold using the printing material. As described herein, the method similarly may be used to fabricate various devices other than a bone-regeneration scaffold using biologically-derived bone powder. Thus, in the following description of the method, references to a “bone-regeneration scaffold” may be replaced with more generally a “device” in accordance with embodiments of the present disclosure.

In some embodiments, the printing material may be a powder or granular formulation. In some embodiments, the printing material may be a slurry. In some embodiments, a concentration of the biologically-derived bone powder in the slurry may be within a range of 55% to 85% by volume. In some embodiments, the slurry also may include a photoinitiator, a dispersant, and a monomer. In some embodiments, the photoinitiator may include diphenyl (2,4,6-trimethylbenzoyl) phosphine oxide. In some embodiments, the dispersant may include Solplus D560. In some embodiments, the monomer may include ethylene glycol dimethacrylate.

In some embodiments, the method also may include preparing the biologically-derived bone powder from one or more biologically-derived bones. In some embodiments, preparing the biologically-derived bone powder from one or more biologically-derived bones may include dissecting the one or more biologically-derived bones from one or more cadavers, removing soft tissues from the one or more biologically-derived bones, soaking the one or more biologically-derived bones in a hydrogen peroxide solution, removing trabecular bone from a distal end and a proximal end of the one or more biologically-derived bones, cutting the one or more biologically-derived bones into a plurality of bone sections, fragmenting the bone sections into bone shavings, grinding the bone shavings into a precursor bone powder, sintering the precursor bone powder to form a sintered bone powder, wet milling the sintered bone powder to form a wet milled bone powder, drying the wet milled bone powder to form a dried bone powder, and dry milling the dried bone powder to form the biologically-derived bone powder.

In some embodiments, the one or more biologically-derived bones may include one or more human bones. In some embodiments, the one or more biologically-derived bones may include one or more animal bones. In some embodiments, soaking the one or more biologically-derived bones in the hydrogen peroxide solution may include soaking the one or more biologically-derived bones in a 6% hydrogen peroxide solution for 30 minutes to kill microorganisms and remaining impurities. In some embodiments, cutting the one or more biologically-derived bones into the plurality of bone sections may include cutting the one or more biologically-derived bones using an orthopedic bandsaw. In some embodiments, fragmenting the bone sections into the bone shavings may include fragmenting the bone sections using a bone mill. In some embodiments, grinding the bone shavings into the precursor bone powder may include grinding the bone shavings using a freezer mill. In some embodiments, sintering the precursor bone powder to form the sintered bone powder may include sintering the precursor bone powder in a muffle furnace. In some embodiments, the precursor bone powder may be sintered with a ramp rate of 2.5° C./min to a holding temperature of 750° C. and held for a dwell time of 2 hours. In some embodiments, wet milling the sintered bone powder to form the wet milled bone powder may include wet milling the sintered bone powder using a planetary ball mill. In some embodiments, the sintered bone powder may be wet milled for 8 hours in 70% ethanol. In some embodiments, drying the wet milled bone powder to form the dried bone powder may include drying the wet milled bone powder using a heat lamp. In some embodiments, dry milling the dried bone powder to form the biologically-derived bone powder may include dry milling the dried bone powder using a planetary ball mill. In some embodiments, the dried bone powder may be dry milled for 2 hours.

In some embodiments, the method also may include preparing the slurry. In some embodiments, preparing the slurry may include mixing a photoinitiator, a dispersant, and a monomer to form a first mixture, and mixing the first mixture and the biologically-derived bone powder to form the slurry. In some embodiments, the photoinitiator may include diphenyl (2,4,6-trimethylbenzoyl) phosphine oxide, the dispersant may include Solplus D560, and the monomer may include ethylene glycol dimethacrylate. In some embodiments, mixing the photoinitiator, the dispersant, and the monomer to form the first mixture may include mixing the photoinitiator, the dispersant, and the monomer using a planetary ball mill.

In some embodiments, the photoinitiator, the dispersant, and the monomer may be mixed within a milling jar containing a plurality of milling balls. In some embodiments, the milling jar may be a yttrium stabilized zirconium planetary ball milling jar, and the milling balls may be yttrium stabilized zirconium milling balls. In some embodiments, the plurality of milling balls may include a plurality of first milling balls each having a first diameter and a plurality of second milling balls each having a second diameter that is greater than the first diameter. In some embodiments, the first diameter may be 5 mm, and the second diameter may be 10 mm. In some embodiments, a ratio of the first milling balls to the second milling balls may be 3:2 by weight %. In some embodiments, a ratio of the milling balls to the biologically-derived bone powder may be 2:1 by weight %.

In some embodiments, mixing the first mixture and the biologically-derived bone powder to form the slurry may include mixing the first mixture and the biologically-derived bone powder using a planetary ball mill. In some embodiments, the first mixture and the biologically-derived bone powder may be mixed within a milling jar containing a plurality of milling balls. In some embodiments, the milling jar may be a yttrium stabilized zirconium planetary ball milling jar, and the milling balls may be yttrium stabilized zirconium milling balls. In some embodiments, the plurality of milling balls may include a plurality of first milling balls each having a first diameter and a plurality of second milling balls each having a second diameter that is greater than the first diameter. In some embodiments, the first diameter may be 5 mm, and the second diameter may be 10 mm. In some embodiments, a ratio of the first milling balls to the second milling balls may be 3:2 by weight %. In some embodiments, a ratio of the milling balls to the biologically-derived bone powder may be 2:1 by weight %.

In some embodiments, mixing the first mixture and the biologically-derived bone powder to form the slurry may include mixing the first mixture and a first amount of the biologically-derived bone powder using a planetary ball mill to form a second mixture, mixing the second mixture and a second amount of the biologically-derived bone powder using the planetary ball mill to form a third mixture, and mixing the third mixture and a third amount of the biologically-derived bone powder using the planetary ball mill to form the slurry. In some embodiments, the first amount may be greater than the second amount, and the second amount may be greater than the third amount. In some embodiments, mixing the first mixture and the first amount of the biologically-derived bone powder using the planetary ball mill to form the second mixture may include mixing the first mixture and the first amount of the biologically-derived bone powder at 300 RPM for 2 hours on a 50% duty cycle. In some embodiments, mixing the second mixture and the second amount of the biologically-derived bone powder using the planetary ball mill to form the third mixture may include mixing the second mixture and the second amount of the biologically-derived bone powder at 320 RPM for 2 hours on a 50% duty cycle. In some embodiments, mixing the third mixture and the third amount of the biologically-derived bone powder using the planetary ball mill to form the slurry may include mixing the third mixture and the third amount of the biologically-derived bone powder at 360 RPM for 2 hours on a 50% duty cycle.

In some embodiments, the bone-regeneration scaffold may be biodegradable. In some embodiments, the bone-regeneration scaffold may be porous. In some embodiments, the bone-regeneration scaffold may include a gyroid structure. In some embodiments, the bone-regeneration scaffold may include a plurality of perfusion channels configured for facilitating perfusion through the bone-regeneration scaffold. In some embodiments, the bone-regeneration scaffold also may include an input port and an output port each in fluid communication with the perfusion channels. In some embodiments, the method also may include treating the perfusion channels with living cells configured for accelerating tissue growth, inhibiting infection, enhancing vascular tissue development, or reducing thrombogenic potential. In some embodiments, the living cells may be autogenic cells. In some embodiments, the method also may include treating the perfusion channels with one or more bioactive agents configured for accelerating tissue growth, inhibiting infection, enhancing vascular tissue development, or reducing thrombogenic potential. In some embodiments, the method also may include treating the bone-regeneration scaffold with one or more osteogenic agents configured for enhancing bone development. In some embodiments, the one or more osteogenic agents may include recombinant bone morphogenic protein. In some embodiments, the one or more osteogenic agents may include vascular endothelial growth factor. In some embodiments, the method also may include comprising treating the bone-regeneration scaffold with patient cellular material. In some embodiments, the patient cellular material may include stem cells. In some embodiments, the patient cellular material may include platelet rich plasma. In some embodiments, the method also may include incubating the bone-regeneration scaffold treated with the patient cellular material. In some embodiments, the method also may include obtaining computed tomography scans of a patient, and the bone-regeneration scaffold may be fabricated based at least in part on the computed tomography scans. In some embodiments, the method also may include obtaining clinician annotations to the computed tomography scans, and the bone-regeneration scaffold may be fabricated based at least in part on the computed tomography scans and the clinician annotations. In some embodiments, a shape of the bone-regeneration scaffold may be based at least in part on a shape of a bone segment to be removed from the patient.

These and other aspects and improvements of the present disclosure will become apparent to one of ordinary skill in the art upon review of the following detailed description when taken in conjunction with the several drawings and the appended claims.

### BRIEF DESCRIPTION OF THE DRAWINGS

FIG. 1 is a perspective view of an example bone-regeneration scaffold in accordance with embodiments of the disclosure, showing the bone-regeneration scaffold having a gyroid structure.

FIG. 2 is a perspective view of another example bone-regeneration scaffold in accordance with embodiments of the disclosure, showing the bone-regeneration scaffold having a gyroid structure.

The detailed description is set forth with reference to the accompanying drawings. The drawings are provided for purposes of illustration only and merely depict example embodiments of the disclosure. The drawings are provided to facilitate understanding of the disclosure and shall not be deemed to limit the breadth, scope, or applicability of the disclosure. The use of the same reference numerals indicates similar, but not necessarily the same or identical components. Different reference numerals may be used to identify similar components. Various embodiments may utilize elements or components other than those illustrated in the drawings, and some elements and/or components may not be present in various embodiments. The use of singular terminology to describe a component or element may, depending on the context, encompass a plural number of such components or elements and vice versa.

### DETAILED DESCRIPTION OF THE DISCLOSURE

In the following description, specific details are set forth describing some embodiments consistent with the present disclosure. Numerous specific details are set forth in order to provide a thorough understanding of the embodiments. It will be apparent, however, to one skilled in the art that some embodiments may be practiced without some or all of these specific details. The specific embodiments disclosed herein are meant to be illustrative but not limiting. One skilled in the art may realize other elements that, although not specifically described here, are within the scope and the spirit of this disclosure. In addition, to avoid unnecessary repetition, one or more features shown and described in association with one embodiment may be incorporated into other embodiments unless specifically described otherwise or if the one or more features would make an embodiment non-functional. In some instances, well known methods, procedures, and/or components have not been described in detail so as not to unnecessarily obscure aspects of the embodiments.

Overview

Embodiments of bone-regeneration scaffolds or other devices composed of biologically-derived bone powder and methods for fabricating such bone-regeneration scaffolds are provided. As described herein, the disclosed methods may be used to fabricate various devices other than a bone-regeneration scaffold using biologically-derived bone powder. Thus, in the following description, references to a “bone-regeneration scaffold” may be replaced with more generally a “device” in accordance with embodiments of the present disclosure. The bone-regeneration scaffolds or other devices can be used in human and veterinary orthopedic medicine. As described herein, a bone-regeneration scaffold may be fabricated as a patient-specific structure by 3D printing and then implanted in a patient for treating a bone defect, such as a critical-sized bone defect. In this manner, the bone-regeneration scaffold or other device may be used to replace a resected region of the patient's bone. In some embodiments, the bone-regeneration scaffold may be used as part of an overall device for treating a bone defect, with the device also including additional components, such as a sleeve configured for protecting the bone-regeneration scaffold and facilitating positioning of the bone-regeneration scaffold relative to bone structures of the patient as well as one or more fixation members configured for attaching to the patient's bone structures. The components of the device may be formed of different materials to provide desired strength and porosity for the various components. Furthermore, the different materials may enable variable degradation rates such that the entire device, including mounting hardware, dissolves or can be removed over time. In some embodiments, the device may be enhanced with bioactive agents to promote bone growth and vascularization. In some embodiments, the device may be connected to host blood supply to accelerate native tissue development after implantation in a patient. In some embodiments, the bone-regeneration scaffolds described herein may be used as a part of the devices described in U.S. application Ser. No. 17/541,121, filed on Dec. 2, 2021, and titled “Methods and Devices for Improving Bone Healing,” the entire disclosure of which is expressly incorporated herein by reference. It will be appreciated, however, that the bone-regeneration scaffolds may be used for various purposes and may be used with or as a part of various types of devices for facilitating bone regeneration.

As described above, certain existing scaffolds for bone regeneration have been formed of ceramic materials, such as HAp and β-TCP, which are highly osteogenic but may lack the structural strength and stiffness required for practical load bearing. Other types of existing scaffolds have been formed of polymeric biomaterials, such as PCL, which provides suitable mechanical strength and elasticity but demonstrates poor osteoconduction and osteoinduction. As described herein, the present bone-regeneration scaffolds may be fabricated from biologically-derived bone powder prepared from human or animal bones. In view of the biologically-derived composition of the bone-regeneration scaffolds described herein, the bone-regeneration scaffolds advantageously may be highly osteogenic while also providing the structural strength and stiffness needed for load bearing applications. The bone-regeneration scaffolds advantageously may be fabricated by 3D printing to have patient-specific shapes and sizes. As described herein, biologically-derived bone powder may be prepared from human or animal cadaver bones and then used in a printing material suitable for 3D printing of the bone-regeneration scaffolds. The printing material may be a powder or granular formulation or a slurry, depending on the 3D printing technique used.

Example Methods, Bone-Regeneration Scaffolds, and Devices

The bone-regeneration scaffolds described herein may be fabricated using biologically-derived bone powder prepared from human or animal cadaver bones. To prepare the biologically-derived bone powder, one or more biologically-derived bones may be dissected from one or more cadavers, and the soft tissues may be removed from the one or more biologically-derived bones. Then, the one or more biologically-derived bones may be soaked in a hydrogen peroxide solution. In some embodiments, the one or more biologically-derived bones may be soaked in a 6% hydrogen peroxide solution for 30 minutes to kill microorganisms and remaining impurities, such as oily fats, degrading the materials and facilitating removal. The trabecular bone at the distal end and the proximal end of the one or more biologically-derived bones may be removed, and the bones may be cut into a plurality of sections. In some embodiments, an orthopedic bandsaw may be used to remove the trabecular bone and cut the bones into bone sections. The bone sections then may be fragmented into bone shavings. In some embodiments, a bone mill and a medium blade may be used to fragment the bone sections into bone shavings. Next, the bone shavings may be ground into a precursor bone powder. In some embodiments, a freezer mill may be used to grind the bone shavings into the precursor bone powder. Next, the precursor bone powder then may be sintered. In some embodiments, the precursor bone powder may be sintered in a muffle furnace with a ramp rate of 2.5° C./min to a holding temperature of 750° C. and held for a dwell time of 2 hours. The sintered bone powder then may be wet milled to form a wet milled bone powder. In some embodiments, the sintered bone powder may be wet milled in a planetary ball mill for 8 hours in 70% ethanol. Next, the wet milled bone powder may be dried to form a dried bone powder. In other words, the ethanol may be evaporated out. In some embodiments, the wet milled bone powder may be dried with a heat lamp. The dried bone powder then may be dry milled to form the biologically-derived bone powder. In some embodiments, the dried bone powder may be dry milled for 2 hours using a planetary ball mill.

The biologically-derived bone powder may be used to prepare a printing material suitable for 3D printing of the bone-regeneration scaffolds. In some embodiments, the printing material may be a powder or granular formulation. In some embodiments, the printing material may be a slurry. Preparation of a slurry generally may include mixing a photoinitiator, a dispersant, and a monomer to form a first mixture, and then mixing the first mixture and the biologically-derived bone powder to form the slurry. In some embodiments, the photoinitiator may be diphenyl (2,4,6-trimethylbenzoyl) phosphine oxide, the dispersant may be Solplus D560 (Lubrizol Advanced Materials Inc.), and the monomer may be ethylene glycol dimethacrylate. Other types of photoinitiators, dispersants, and monomers may be used in other embodiments. In some embodiments, the photoinitiator, the dispersant, and the monomer may be mixed using a planetary ball mill. The photoinitiator, the dispersant, and the monomer may be mixed within a milling jar containing a plurality of milling balls. In some embodiments, the milling jar may be a yttrium stabilized zirconium planetary ball milling jar, and the milling balls may be yttrium stabilized zirconium milling balls. In some embodiments, the plurality of milling balls may include a plurality of first milling balls each having a first diameter and a plurality of second milling balls each having a second diameter that is greater than the first diameter. In some embodiments, the first diameter may be 5 mm, and the second diameter may be 10 mm. In some embodiments, a ratio of the first milling balls to the second milling balls may be 3:2 by weight %. In some embodiments, a ratio of the milling balls to the biologically-derived bone powder may be 2:1 by weight %. In some embodiments, the first mixture and the biologically-derived bone powder may be mixed using the same planetary ball mill, milling jar, and milling balls.

In some embodiments, the first mixture and a first amount of the biologically-derived bone powder may be mixed using the planetary ball mill to form a second mixture, the second mixture and a second amount of the biologically-derived bone powder may be mixed using the planetary ball mill to form a third mixture, and then the third mixture and a third amount of the biologically-derived bone powder may be mixed using the planetary ball mill to form the slurry. In some embodiments, the first amount may be greater than the second amount, and the second amount may be greater than the third amount. In some embodiments, the first mixture and the first amount of the biologically-derived bone powder may be mixed at 300 RPM for 2 hours on a 50% duty cycle. In some embodiments, the second mixture and the second amount of the biologically-derived bone powder may be mixed at 320 RPM for 2 hours on a 50% duty cycle. In some embodiments, the third mixture and the third amount of the biologically-derived bone powder may be mixed at 360 RPM for 2 hours on a 50% duty cycle. In some embodiments, a concentration of the biologically-derived bone powder in the slurry may be within a range of 25% to 85% by volume or within a range of 55% to 85% by volume.

In one specific example conducted, biologically-derived canine bone powder, prepared in accordance with the method described above, was used to produce 10 mL of a high solid loading canine bone powder slurry (with a canine bone powder concentration within a range of 25.0% to 85.0% by volume) suitable for 3D printing a bone-regeneration scaffold. The slurry was prepared using 32.180 g of canine bone powder, 0.024 g of diphenyl (2,4,6-trimethylbenzoyl) phosphine oxide (TCI America) as the photoinitiator, 0.973 mL of Solplus D560 (Lubrizol Advanced Materials Inc.) as the dispersant, and 5.0 mL of ethylene glycol dimethacrylate (Scientific Polymer Products Inc.) as the monomer. A pair of 50 mL yttrium stabilized zirconium planetary ball milling jars were used, with each of the milling jars containing a plurality of 5 mm diameter (“small”) yttrium stabilized zirconium milling balls and a plurality of 10 mm diameter (“large”) yttrium stabilized zirconium milling balls. The ratio of the milling balls to the canine bone powder was 2:1 by weight %, and the ratio of the small milling balls to the large milling balls was 3:2 by weight %.

A 10 mL graduated cylinder was used to measure the monomer and pour the measured amount into each of the milling jars. Next, a 1 mL plastic syringe was used to measure the dispersant and add the measured amount to each of the milling jars. An analytical scale, weigh paper, and a weigh boat were used to measure the photoinitiator and add the measured amount to each of the milling jars. The analytical scale, weigh paper, and a weigh boat were used to measure 15.180 g of the canine bone powder for each of the milling jars, which was then set aside. Based on the weight of the canine bone powder, the milling balls were selected and weighed to provide a ratio of the milling balls to the canine bone powder of 2:1 by weight % and a ratio of the small milling balls to the large milling balls of 3:2 by weight %. The measured milling balls were then added to each of the milling jars, and the milling jar lids were sealed using parafilm and laboratory tape. Next, the milling jars were placed in a planetary ball mill, on opposite sides to keep the instrument balanced, and fastened with the securing apparatuses that accompany the planetary ball mill. The photoinitiator, the dispersant, and the monomer were mixed at 120 RPM for 30 minutes on a 50% duty cycle (3 cycles of 5 minutes on and 5 minutes off) to form a first mixture, and the milling jars were then removed from the planetary ball mill. Next, the measured canine bone powder previously set aside was placed in a ceramic mortar and ground using a ceramic pestle until no clumps remained. The ground canine bone powder was re-weighed and added to each of the milling jars (15.180 g/jar). Using a laboratory spatula, for each of the milling jars, the components were mixed by hand until there was no dry powder left in the mixture, and then the milling jar lids were sealed using parafilm and laboratory tape. Then, the milling jars were installed and fastened, opposite one another, in the planetary ball mill, and the components were mixed at 300 RPM for 2 hours on a 50% duty cycle (12 cycles of 5 minutes on and 5 minutes off) to form a second mixture.

Next, the analytical scale, weigh paper, and a weigh boat were used to measure 10.0 g of the canine bone powder for each of the milling jars, and the measured canine bone powder was ground using the ceramic mortar and pestle until no clumps remained. The ground canine bone powder was re-weighed and added to each of the milling jars (10.0 g/jar). Using a laboratory spatula, for each of the milling jars, the components were mixed by hand until there was no dry powder left in the mixture, and then the milling jar lids were sealed using parafilm and laboratory tape. Next, the milling jars were installed and fastened, opposite one another, in the planetary ball mill, and the components were mixed at 320 RPM for 2 hours on a 50% duty cycle (12 cycles of 5 minutes on and 5 minutes off) to form a third mixture.

Next, the analytical scale, weigh paper, and a weigh boat were used to measure 7.0 g of the canine bone powder for each of the milling jars, and the measured canine bone powder was ground using the ceramic mortar and pestle until no clumps remained. The ground canine bone powder was re-weighed and added to each of the milling jars (7.0 g/jar). Using a laboratory spatula, for each of the milling jars, the components were mixed by hand until there was no dry powder left in the mixture, and then the milling jar lids were sealed using parafilm and laboratory tape. Next, the milling jars were installed and fastened, opposite one another, in the planetary ball mill, and the components were mixed at 360 RPM for 2 hours on a 50% duty cycle (12 cycles of 5 minutes on and 5 minutes off) to form the slurry. On the next day, the slurry was transferred to luer tip syringes. The milling jars were retrieved and opened. The milling balls were collected with tongs, and the slurry was mixed manually for 5-10 minutes. The slurry was visually inspected to confirm that no clusters of powder were present, and observations were recorded with respect to the color, texture, viscosity, and homogeneity of the slurry. In the event that the slurry is not homogenous, the slurry may be missed manually for an additional 5 minutes and further observations may be recorded. The walls of the jars were cleaned with a spatula, as all of the slurry was transferred to the luer tip syringes before washing the milling jars. Each of the syringes was closed at the tip with a stopper and wrapped with aluminum foil.

Although the preceding example describes use of biologically-derived canine bone powder to prepare a canine bone powder slurry, it will be appreciated that the described techniques are not limited to canine applications. Indeed, the described techniques may be applied using a biologically-derived human bone powder to prepare a human bone powder slurry or using any biologically-derived animal bone powder to prepare an animal bone powder slurry.

A printing material, such as a power or granular formulation or a slurry, prepared according to the methods described above may be used for 3D printing a bone-regeneration scaffold. It will be appreciated that bone-regeneration scaffolds are just an example of an object that can be produced using biologically-derived bone powder as described herein. The biologically-derived bone powder also may be used to produce other types of scaffolds or other types of objects that may aid in bone regeneration but for other reasons, such as stiffeners to provide structural support or other types of fixation devices. As described above, an advantage of the biologically-derived bone powder described herein is that would ultimately degrade and would be remodeled by the patient into natural host (endogenous) bone.

Various types of additive manufacturing (AM) techniques may be used for fabricating a bone-regeneration scaffold or other types of scaffolds or objects from a printing material including the biologically-derived bone powder using a 3D printer. Example AM techniques include, but are not limited to, directed energy deposition, powder bed fusion, binder jetting, melt extrusion, viscous extrusion, photopolymer-based AM, and material jetting. Directed energy deposition may use one or more powder formulations based on the biologically-derived bone powder and/or derivatives thereof to fabricate objects. Powder bed fusion may use one or more powder or otherwise granular formulations of the biologically-derived bone powder to fabricate objects. Binder jetting may use one of more powder or otherwise granular formulations of the biologically-derived bone powder to fabricate objects using any number of binders that are commonly used or could be used in this AM method to harden the powder material into objects. Melt extrusion may incorporate one of more powder formulations of the biologically-derived bone powder into polymeric filament that could be thermally extruded into objects that could be composed in large part of the biologically-derived bone powder. Such objects could be sintered to remove the polymeric content and consolidate/densify the object. Viscous extrusion may incorporate one of more powder formulations of the biologically-derived bone powder into a viscous material such as hydrogel, biogel, alginate, Pluronic or other similar material that is commonly used in this AM method, and which could be extruded into shapes that could be hardened into objects using any of the many methods of doing this in this AM method. Photopolymer-based AM may use one of more powder formulations of the biologically-derived bone powder combined with photopolymeric components such as photoinitiator, monomer, dispersant, etc. in either inverted or vat-based AM methods, and may employ DLP, LCD, laser, or any other photopolymerization energy source. Material jetting may incorporate one of more powder formulations of the biologically-derived bone powder into polymeric materials that could be jetted into objects. Still other types of AM techniques, currently existing or later developed, may be used for fabricating a bone-regeneration scaffold or other types of scaffolds or objects from a printing material including the biologically-derived bone powder using a 3D printer.

In some embodiments, the bone-regeneration scaffold may be fabricated based at least in part on surgical computed tomography (CT) scans of a patient taken pre-operatively. In some embodiments, a surgeon may indicate on the CT direct locations in which surgical incisions should be conducted on the patient. The surgeon may also indicate ideal locations for screw attachment points, the desired dimensions of screws (e.g., custom screw size, length, etc.), and appropriate landmarks for screw guide registration, which may be derived from the surgical CT scan imagery. Surgical CT scan imagery with annotations may subsequently be delivered (e.g., digitally or otherwise) to a laboratory for manufacture of a 3D printed patient-specific bone-regeneration scaffold as well as other patient-specific components of the overall device to be implanted in the patient. Furthermore, the surgeon may identify bone landmarks for the development of 3D printed surgical cutting guides from the surgical CT scan imagery that may be produced in tandem with the device to assist in later implantation. The procedures described here could be completed on single-ended or dual-ended implants for treating bone defects or for various other types of surgical cases, such as mandibular repair. The laboratory may then manufacture the device, which may be a 3D printed porous rigid implant for large defect repair in long bones (including, but not limited to applications in which the defect is load-bearing), directly from the surgical CT scan imagery of the patient, as provided from the surgeon.

In some embodiments, the laboratory may 3D print one or more 3D surgical cutting guides to directly correspond with the patient bone interface. The 3D surgical cutting guides may register the implant with the host bone location via the indicated bone landmarks as annotated by the surgeon. In some embodiments, the laboratory may 3D print a bone-regeneration scaffold and associated components of the device in two material types: (1) a softer, more porous material that tunably degrades in controlled timeframes, such as within 4 to 6 months, or as desired; or (2) a hard, less porous material that tunably degrades in controlled timeframes, such as within 1 to 2 years, or as desired.

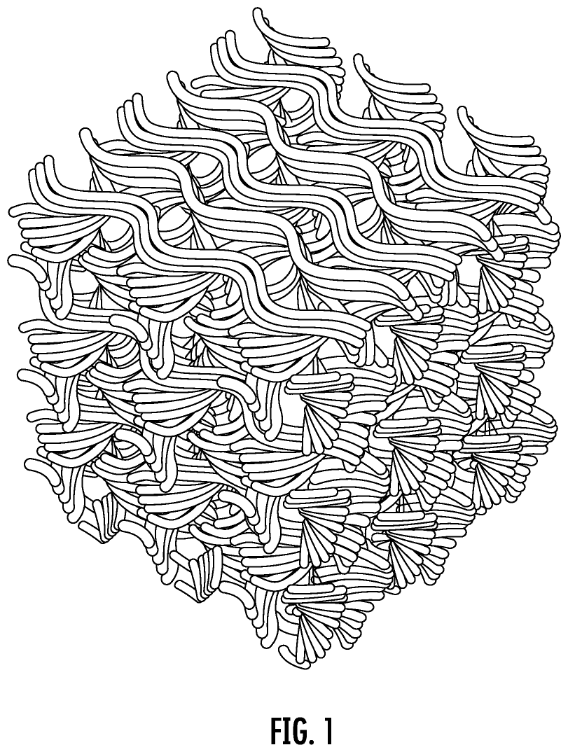

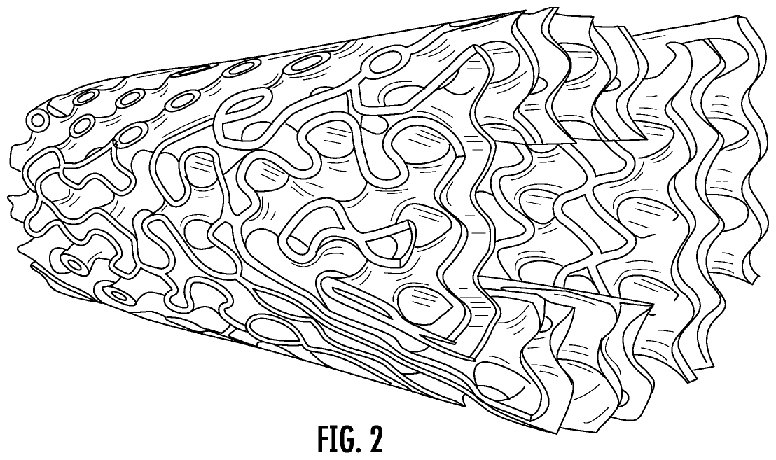

The bone-regeneration scaffold may be 3D printed in the softer, more porous material. FIGS. 1 and 2 illustrate non-limiting examples of a bone-regeneration scaffold in accordance with embodiments of the present disclosure. It will be appreciated, however, that bone-regeneration scaffolds having various shapes and configurations may be fabricated in accordance with the teachings of the present disclosure. As discussed above, the bone-regeneration scaffold may be 3D printed to correspond to the shape of the host bone or bone segment which is to be removed and replaced. In some embodiments the bone-regeneration scaffold may have a gyroid structure. Various other types of structures, such as rectilinear structures or diamond unit cell structures, may be used in other embodiments. In some embodiments the bone-regeneration scaffold may include a plurality of fine, internal perfusion channels configured to enhance perfusion through the bone-regeneration scaffold. In some embodiments, the perfusion channels may be formed by interconnected pores. In some embodiments, the bone-regeneration scaffold may include an input port and an output port each in fluid communication with the perfusion channels. For example, the perfusion channels may converge to a single input port and a single output port, which may protrude from the bone-regeneration scaffold. In some embodiments, the bone-regeneration scaffold may include one or more channels for insertion of 3D printed rods therein and/or attachment of 3D printed rods thereto. In some embodiments, one or more screw attachment collars may be 3D printed for attaching the bone-regeneration scaffold to the outer cortex of the patient's bone. The screw attachment collars may be 3D printed in the softer, more porous material.

In some embodiments, additional components of the overall device may be 3D printed in the harder, less porous material. These components may include one or more drilling guides, one or more rods, one or more anchors, and one or more screws. The drilling guide may be configured for guiding placement of the screws for securing the device to the patient's bone. The rods may be any cross-sectional profile (e.g., I-beam or cylindrical) and may be configured for increasing stiffness of the device. The anchors may include channels into which the rods may be inserted, which may provide additional structural support for the rods in the host bone medullar cavity. In some embodiments, these components may be made from biologically-derived bone powder, but with different (biocompatible) polymeric additives (e.g., photopolymeric materials that may vary the properties of the components). It is envisioned that any animal bone or soft tissue, such as cartilage, may be processed and used either in place of or in addition to cadaver bone.

In some embodiments, the laboratory may 3D print the various components according to the surgical CT scan imagery data and the surgeon's bone landmark annotations and/or other instructions. For example, screw holes in the guide may be configured to align with holes in the anchors and implant body attachment tabs. In some embodiments, the laboratory may assemble the device in sterile and/or sanitized conditions, e.g., inserting and attaching the rods into the bone-regeneration scaffold, using a biocompatible adhesive that may degrade in approximately one year, or within a desired time range, etc. In some embodiments, the laboratory may treat the perfusion channels of the bone-regeneration scaffold with autogenic cells and/or bioactive agents to resist infection, enhance vascular tissue development, and/or reduce thrombogenic potential, among other potential therapeutic applications. In some embodiments, the laboratory may treat the device with osteogenic agents to accelerate or enhance bone development on or within the bone-regeneration scaffold, including, but not limited to, recombinant human bone morphogenic protein and/or vascular endothelial growth factor, among others. In some embodiments, the laboratory may seed patient cellular material (e.g., stem cells) and milieu, and may “grow” the device in an incubator for a specified amount of time (e.g., approximately one week). In some embodiments, the laboratory may sterilize remaining associated components (where applicable) and package the device, including the “live” bone-regeneration scaffold, for delivery to the operating room.

During implantation of the bone-regeneration scaffold and the overall device for treating a bone defect, the surgeon may use the 3D printed surgical cutting guides to guide in the removal of any corresponding segment of bone that may be harboring a tumor or lesion from the patient. In some embodiments, the surgeon may implant the anchor(s) of the device into the end(s) of the host bone, and press fit the device into the medullary canal. In some embodiments, the surgeon may place the drilling guide over the exposed end of the host bone, aligning it with a notch in the anchor, and may subsequently drill holes into the host cortical bone to enable accurate screw placement. In some embodiments, the surgeon may connect the regional host blood supply directly to the perfusion input port and output port on the device to provide host blood supply. In some embodiments, the surgeon may install the device by aligning and introducing the rods into the channels in the anchor(s), which may be installed in the patient host bone at one or both ends, as required. In some embodiments, the surgeon may install the screws in the device attachment points in the screw attachment collar to provide additional structural support. In some embodiments, the screws may proceed through the entire device and host bone, specifically, through the host cortical bone, through the anchor, through the opposite side host bone, and into the opposite side screw attachment collar. In some embodiments, the materials of the components of the device may degrade completely, depending on a designed/desired timeframe, and may be tunable based on direct application within the device and patient needs.

The methods, bone-regeneration scaffolds, and devices described herein may enable complete bone healing and removal of fixation, leaving the patient with only endogenous bone as in the Franch study (see Franch J. et al. Use of three-dimensionally printed β-tricalcium phosphate synthetic bone graft combined with recombinant human bone morphogenic protein-2 to treat a severe radial atrophic nonunion in a Yorkshire terrier. Veterinary Surgery 2020:vsu.13476. https://doi.org/10.1111/vsu.13476), after the endoprostheses device degrades safely in the body. The devices described herein may also provide a platform for delivery of an antibiotic or any bioactive agent from the PCL and/or β-TCP (see Chang H-I. et al. Controlled release of an antibiotic, gentamicin sulphate, from gravity spun polycaprolactone fibers. J Biomed Mater Res 2008; 84A:230-7. https://doi.org/10.1002/jbm.a.31476), use of VEGF to accelerate angiogenesis deep in the scaffold (see Park, JiSun et al. Fabrication and characterization of 3D-printed bone-like β-tricalcium phosphate/polycaprolactone scaffolds for dental tissue engineering. Journal of Industrial and Engineering Chemistry 2017; 46:175-81. https://doi.org/10.1016/j.jiec.2016.10.028; Yan Y. et al. Vascularized 3D printed scaffolds for promoting bone regeneration. Biomaterials 2019; 190-191:97-110. https://doi.org/10.1016/j.biomaterials.2018.10.033), functionally graded scaffold topology to optimize permeability, osteoinduction and scaffold strength, embedded telemetry, strain gauges, and/or MEMs.

The methods, bone-regeneration scaffolds, and devices provided herein may be used for both animal and human health applications. Sheep have been utilized to model the disclosed methods, bone-regeneration scaffolds, and devices, as sheep are a highly suitable model for the human clinical situation with similarities in weight and bone dimensions, macro- and microstructure, mineral composition, biomechanics and remodeling (see Pearce, A. et al. Animal models for implant biomaterial research in bone: A review. ECM 2007; 13:1-10. https://doi.org/10.22203/eCM.v013a01; Reichert J C. et al. A Tissue Engineering Solution for Segmental Defect Regeneration in Load-Bearing Long Bones. Science Translational Medicine 2012; 4:141ra93-141ra93. https://doi.org/10.1126/scitranslmed.3003720). These similarities enable medical devices for human orthopedics to be used in sheep (see Newman E. et al. The Potential of Sheep for the Study of Osteopenia: Current Status and Comparison with Other Animal Models n.d.; 16:8). Materials used for the bone-regeneration scaffolds and human bone powder from AlloSource have been thoroughly studied and known to be biocompatible for implantation in live animals and FDA approved for numerous applications.

Although specific embodiments of the disclosure have been described, one of ordinary skill in the art will recognize that numerous other modifications and alternative embodiments are within the scope of the disclosure. Further, while various illustrative implementations have been described in accordance with embodiments of the disclosure, one of ordinary skill in the art will appreciate that numerous other modifications to the illustrative implementations described herein are also within the scope of this disclosure.

Although embodiments have been described in language specific to structural features and/or methodological acts, it is to be understood that the disclosure is not necessarily limited to the specific features or acts described. Rather, the specific features and acts are disclosed as illustrative forms of implementing the embodiments. Conditional language, such as, among others, “can,” “could,” “might,” or “may,” unless specifically stated otherwise, or otherwise understood within the context as used, is generally intended to convey that certain embodiments could include, while other embodiments do not include, certain features, elements, and/or steps. Thus, such conditional language is not generally intended to imply that features, elements, and/or steps are in any way required for one or more embodiments.

1. A method for fabricating a bone-regeneration scaffold, the method comprising:
providing a printing material comprising a biologically-derived bone powder; and
fabricating, via a 3D printer, the bone-regeneration scaffold using the printing material.

2. (canceled)

3. The method of claim 1, wherein the printing material is a slurry.

4. (canceled)

5. The method of claim 3, wherein the slurry further comprises a photoinitiator, a dispersant, and a monomer.

6. The method of claim 5, wherein the photoinitiator comprises diphenyl (2,4,6-trimethylbenzoyl) phosphine oxide.

7. The method of claim 5, wherein the dispersant comprises Solplus D560.

8. The method of claim 5, wherein the monomer comprises ethylene glycol dimethacrylate.

9. (canceled)

10. The method of claim 1, further comprising:
preparing the biologically-derived bone powder from one or more biologically-derived bones, wherein preparing the biologically-derived bone powder from the one or more biologically-derived bones comprises:
dissecting the one or more biologically-derived bones from one or more cadavers;
removing soft tissues from the one or more biologically-derived bones;
soaking the one or more biologically-derived bones in a hydrogen peroxide solution;
removing trabecular bone from a distal end and a proximal end of the one or more biologically-derived bones;
cutting the one or more biologically-derived bones into a plurality of bone sections;
fragmenting the bone sections into bone shavings;
grinding the bone shavings into a precursor bone powder;
sintering the precursor bone powder to form a sintered bone powder;
wet milling the sintered bone powder to form a wet milled bone powder;
drying the wet milled bone powder to form a dried bone powder; and
dry milling the dried bone powder to form the biologically-derived bone powder.

11.-17. (canceled)

18. The method of claim 8, wherein the precursor bone powder is sintered with a ramp rate of 2.5° C./min to a holding temperature of 750° C. and held for a dwell time of 2 hours, the sintered bone powder is wet milled for 8 hours in 70% ethanol, and the dried bone powder is dry milled for 2 hours.

19.-24. (canceled)

25. The method of claim 3, further comprising preparing the slurry, wherein preparing the slurry comprises:
mixing a photoinitiator, a dispersant, and a monomer to form a first mixture;
mixing the first mixture and the biologically-derived bone powder to form the slurry.

26. The method of claim 25, wherein the photoinitiator comprises diphenyl (2,4,6-trimethylbenzoyl) phosphine oxide, wherein the dispersant comprises Solplus D560, and wherein the monomer comprises ethylene glycol dimethacrylate.

27. (canceled)

28. The method of claim 26, wherein the photoinitiator, the dispersant, and the monomer are mixed within a milling jar containing a plurality of milling balls,
wherein the milling jar is a yttrium stabilized zirconium planetary ball milling jar, and wherein the milling balls are yttrium stabilized zirconium milling ball, and
wherein the plurality of milling balls comprises a plurality of first milling balls each having a first diameter and a plurality of second milling balls each having a second diameter that is greater than the first diameter.

29. (canceled)

30. (canceled)

31. The method of claim 30, wherein the first diameter is 5 mm, wherein the second diameter is 10 mm, wherein a ratio of the first milling balls to the second milling balls is 3:2 by weight %; and wherein a ratio of the milling balls to the biologically-derived bone powder is 2:1 by weight.

32. (canceled)

33. (canceled)

34. The method of claim 25, wherein mixing the first mixture and the biologically-derived bone powder to form the slurry comprises mixing the first mixture and the biologically-derived bone powder using a planetary ball mill,
wherein the first mixture and the biologically-derived bone powder are mixed within a milling jar containing a plurality of milling balls;
wherein the milling jar is a yttrium stabilized zirconium planetary ball milling jar, and wherein the milling balls are yttrium stabilized zirconium milling balls; and
wherein the plurality of milling balls comprises a plurality of first milling balls each having a first diameter and a plurality of second milling balls each having a second diameter that is greater than the first diameter.

35.-37. (canceled)

38. The method of claim 37, wherein the first diameter is 5 mm, wherein the second diameter is 10 mm, wherein a ratio of the first milling balls to the second milling balls is 3:2 by weight % and wherein a ratio of the milling balls to the biologically-derived bone powder is 2:1 by weight %.

39. (canceled)

40. (canceled)

41. The method of claim 25, wherein mixing the first mixture and the biologically-derived bone powder to form the slurry comprises:
mixing the first mixture and a first amount of the biologically-derived bone powder using a planetary ball mill to form a second mixture;
mixing the second mixture and a second amount of the biologically-derived bone powder using the planetary ball mill to form a third mixture;
mixing the third mixture and a third amount of the biologically-derived bone powder using the planetary ball mill to form the slurry;
wherein the first amount is greater than the second amount, and wherein the second amount is greater than the third amount.

42.-48. (canceled)

49. The method of claim 1, wherein the bone-regeneration scaffold comprises a plurality of perfusion channels configured for facilitating perfusion through the bone-regeneration scaffold; and
wherein the bone-regeneration scaffold further comprises an input port and an output port each in fluid communication with the perfusion channels.

50. (canceled)

51. The method of claim 49, further comprising treating the perfusion channels with:
living autogenic cells configured for accelerating tissue growth, inhibiting infection, enhancing vascular tissue development, or reducing thrombogenic potential;
one or more bioactive agents configured for accelerating tissue growth, inhibiting infection, enhancing vascular tissue development, or reducing thrombogenic potential.

52. (canceled)

53. (canceled)

54. The method of claim 1, further comprising treating the bone-regeneration scaffold with one or more osteogenic agents configured for enhancing bone development;
wherein the one or more osteogenic agents comprises recombinant bone morphogenic protein and/or vascular endothelial growth factor.

55. (canceled)

56. (canceled)

57. The method of claim 1, further comprising treating the bone-regeneration scaffold with patient cellular material,
wherein the patient cellular material comprises stem cells and/or platelet rich plasma.

58.-60. (canceled)

61. The method of claim 1, further comprising:
obtaining computed tomography scans of a patient, wherein the bone-regeneration scaffold is fabricated based at least in part on the computed tomography scans,
obtaining clinician annotations to the computed tomography scans, wherein the bone-regeneration scaffold is fabricated based at least in part on the computed tomography scans and the clinician annotations;
wherein a shape of the bone-regeneration scaffold is based at least in part on a shape of a bone segment to be removed from the patient.

62.-70. (canceled)
